(19) United States
(12) Patent Application Publication
(10) Pub. No.: US 2025/0129198 A1
(43) Pub. Date: Apr. 24, 2025

(54) PROCESS FOR THE PREPARATION OF A BONDING RESIN

(71) Applicant: Stora Enso OYJ, Helsinki (FI)

(72) Inventors: Ashar Zafar, Älta (SE); Dimitri Areskogh, Bromma (SE); Huynh Tram Anh Pham, Älvsjö (SE)

(73) Assignee: Stora Enso OYJ, Helsinki (FI)

(21) Appl. No.: 18/722,211

(22) PCT Filed: Dec. 19, 2022
(86) PCT No.: PCT/IB2022/062449

§ 371 Date: Jun. 20, 2024

(30) Foreign Application Priority Data
Dec. 21, 2021 (SE) 2151565-5

(51) Int. Cl. C08G 16/02; C04B 26/12; C08K 7/14; C08L 97/02; C09J 161/00

(52) U.S. Cl. CPC C08G 16/0225; C04B 26/12; C08K 7/14; C08L 97/02; C09J 161/00

(57) ABSTRACT

The present invention relates to a bonding resin useful for example in the manufacture of laminates, mineral wool insulation and wood products such as plywood, oriented strandboard (OSB), laminated veneer lumber (LVL), medium density fiber-boards (MDF), high density fiberboards (HDF), parquet flooring, curved plywood, veneered particleboards, veneered MDF or particle boards. The bonding resin is also useful for example in composites, molding compounds and foundry applications. The invention also relates to a method for preparing the bonding resin. The bonding resin comprises hydroxymethylfurfural, furfural, furfuryl alcohol, acetoxymethyl furfural or oligomer of hydroxymethylfurfural or a combination thereof and one or more crosslinker and optionally one or more additives.

### FIELD OF THE INVENTION

The present invention relates to a bonding resin useful for example in the manufacture of laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The bonding resin is also useful for example in composites, molding compounds and foundry applications. The invention also relates to a method for preparing the bonding resin.

### BACKGROUND

When manufacturing wood products such as plywood and LVL, an adhesive formulation is typically formulated by adding fillers and extenders to phenolic resin to provide holdout on the surface, control the rheology for the specific application method and make the adhesive cheaper. The adhesive formulation is typically formulated by mixing phenolic resin, hardener, filler and/or water in a certain ratio.

One problem when preparing an adhesive formulation is to ensure that adequate properties are achieved, particularly the mechanical performance of the wood product manufactured using the adhesive formulation. At the same time, it is desirable to use as much renewable material as possible in the adhesive formulation and at the same time minimize the use of for example phenol and/or formaldehyde.

U.S. Pat. No. 2,619,475 is directed to glass fiber structures using a binder comprising furfuryl alcohol, carbamate of starch and an acidic catalyst.

US2020199022A1 is directed to an aqueous sizing composition used for mineral or organic fibers and comprising a furan resin and a reducing or non-reducing sugar.

### SUMMARY OF THE INVENTION

It has now surprisingly been found that it is possible to easily prepare a bonding resin in which the use of formaldehyde can be avoided. It has also been found that a bonding resin can be achieved without the use of lignin.

It has been found that by using hydroxymethylfurfural (HMF), furfural (FU), furfuryl alcohol (FA), acetoxymethyl furfural or an oligomer of HMF or a combination thereof, a bio-based thermoset binder can be achieved, also without the use of lignin.

It has been found that the bonding resin according to the present invention speeds up the curing reaction significantly and hence reduces the pressing time and enables the use of a lower pressing temperature for curing the bonding resin, when manufacturing for example laminates, mineral wool insulation, glass wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The bonding resin is also useful for example in composites, molding compounds and foundry applications.

The present invention is thus directed to a method for preparing a bonding resin, wherein an aqueous solution of hydroxymethylfurfural (HMF), furfural (FU), furfuryl alcohol (FA), acetoxymethyl furfural or an oligomer of HMF or a combination thereof is mixed with one or more crosslinker selected from glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound (such as tannin, cardanol, cardol, anacardic acid) or epoxidized plant-based oil (such as rapeseed oil, linseed oil, soy bean oil), tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate and optionally one or more additives.

The present invention is thus also directed to the bonding resin obtainable using the method described herein and to the use of the bonding resin in the manufacture of laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards. The present invention is also directed to such laminates, mineral wool insulation and wood products such as plywood, oriented strandboard (OSB), laminated veneer lumber (LVL), medium density fiberboards (MDF), high density fiberboards (HDF), parquet flooring, curved plywood, veneered particleboards, veneered MDF or particle boards manufactured using the bonding resin. The bonding resin according to the present invention may also be used in the manufacture of composites, molding compounds and foundry applications. The bonding resin is also useful in the manufacture of wood fiber inulsation, coating formulations for metal and wood, moulding compounds and fiber reinforced composites.

Thus, one aspect of the present invention is a bonding resin comprising an aqueous solution of hydroxymethylfurfural (HMF), furfural (FU), furfuryl alcohol (FA), acetoxymethyl furfural or an oligomer of HMF and one or more crosslinker selected from glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate; and optionally one or more additives.

### DETAILED DESCRIPTION

The hydroxymethylfurfural (HMF), furfural (FU), furfuryl alcohol (FA) or acetoxymethyl furfural is preferably provided in liquid form, preferably as an aqueous solution. In one embodiment, the aqueous solution comprising hydroxymethylfurfural (HMF), furfural (FU), furfuryl alcohol (FA) or acetoxymethyl furfural also comprises base, preferably ammonia and/or an organic base. Preferably, hydroxymethylfurfural is used according to the present invention. HMF oligomers are compounds having at least two linked HMF units/monomers. HMF oligomers preferably have a molar mass up to 3000 g/mol. HMF oligomers can be prepared according to methods known in the art, for example through a polycondensation.

The glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound (such as tannin, cardanol, cardol, anacardic acid) or epoxidized plant-based oil (such as rapeseed oil, linseed oil, soy bean oil), tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, and/or diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate that is used according to the present invention acts as a cross-linker. Glycidyl ethers with more functional epoxide groups can be used such as glycerol diglycidyl ether, glycerol triglycidyl ether and sorbitol polyglycidyl ether. Other glycidyl ethers having two to nine alkylene glycol groups (such as 2-4 alkylene glycol groups or 2-6 alkylene glycol groups) can be used, such as diethylene glycol diglycidyl ether, triethylene glycol diglycidyl ether, dipropylene glycol diglycidyl ether and tripropylene diglycidyl ether. Other suitable crosslinkers include crosslinkers having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate and polyglycidyl methacrylate. Typically, the bonding resin according to the present invention is applied to the surfaces of for example veneers, such as in the manufacture of plywood. When the veneers are pressed together under heating, the cross-linking in the bonding resin takes place, resulting in an adhesive.

In one embodiment of the present invention, the cross-linker has an epoxy index above 4 eq/kg. The epoxy index can be determined according to ISO 3001. Preferably, the cross-linker has an epoxy index above 5 eq/kg.

An aqueous solution of hydroxymethylfurfural (HMF), furfural (FU), furfuryl alcohol (FA), acetoxymethyl furfural or an oligomer of HMF can be prepared using methods known in the art, such as by mixing hydroxymethylfurfural (HMF), furfural (FU), furfuryl alcohol (FA) or acetoxymethyl furfural and water to obtain a solution. The pH of the aqueous solution of hydroxymethylfurfural (HMF), furfural (FU), furfuryl alcohol (FA) or acetoxymethyl furfural may be in the range of from 4 to 10 or from 6 to 8 or may optionally be adjusted so that the pH is in the range of from 6 to 14 or from 8 to 14, more preferably in the range of from 10 to 14. Such pH adjustment is preferably carried out by addition of base. The base may be alkali or an inorganic base or preferably ammonia and/or organic base. Examples of organic bases include amines, such as primary, secondary and tertiary amines and mixtures thereof. Preferably, the organic base, if used, is selected from the group consisting of methylamine, ethylamine, propylamine, butylamine, ethylenediamine, methanolamine, ethanolamine, aniline, cyclohexylamine, benzylamine, dimethylamine, diethylamine, dipropylamine, dibutylamine, dimethanolamine, diethanolamine, diphenylamine, phenylmethylamine, phenylethylamine, dicyclohexylamine, piperazine, imidazole, 2-methylimidazole, 2-ethylimidazole, 2-ethyl-4-methylimidazole, 2-isopropylimidazole, 2-phenylimidazole, 2-methylimidazoline, 2-phenylimidazoline, trimethylamine, triethylamine, dimethylhexylamine, N-methylpiperazine, dimethylbenzylamine, aminomethyl propanol, tris(dimethylaminomethyl)phenol and dimethylaniline or mixtures thereof. The total amount of ammonia and/or organic base in the aqueous solution is preferably in the range of from 0.1 wt-% to 20 wt-%, preferably 0.1 wt-% to 10 wt-%, of the total weight of the aqueous solution comprising water, ammonia and/or an organic base and crosslinker.

The amount of hydroxymethylfurfural (HMF), furfural (FU), furfuryl alcohol (FA), acetoxymethyl furfural or oligomer of HMF or combination thereof in the aqueous solution is preferably from 1 wt-% to 70 wt-% of the solution, such as from 10 wt-% to 50 wt-% of the solution, based on the dry weight of hydroxymethylfurfural (HMF), furfural (FU), furfuryl alcohol (FA), acetoxymethyl furfural or an oligomer of HMF or combination thereof and the total weight of the solution.

Thus, in a bonding resin according to the present invention the hydroxymethylfurfural (HMF), furfural (FU), furfuryl alcohol (FA), acetoxymethyl furfural or oligomer of HMF is dissolved.

The weight ratio between hydroxymethylfurfural (HMF), furfural (FU), furfuryl alcohol (FA), acetoxymethyl furfural or oligomer of HMF (dry weight) and the total amount of crosslinker is preferably in the range of from 0.1:10 to 10:0.1, such as from 1:10 to 10:0.3, such as from 5:10 to 5:0.3, such as from 1:10 to 10:1. The amount of hydroxymethylfurfural (HMF), furfural (FU), furfuryl alcohol (FA) or acetoxymethyl furfural in the bonding resin is preferably from 5 wt-% to 50 wt-%, calculated as the dry weight of hydroxymethylfurfural (HMF), furfural (FU), furfuryl alcohol (FA) or acetoxymethyl furfural and the total weight of the bonding resin.

The solid content of the bonding resin is preferably up to 80%, such as in the range of from 1 to 60% or in the range of from 15 to 40%.

The bonding resin may also comprise additives, such as urea, tannin, surfactants, dispersing agents and fillers. The bonding resin may also comprise plasticizer. The bonding resin may also comprise coupling agent. Coupling agents are for example silane-based coupling agents.

A filler and/or hardener can also be added to the bonding resin. Examples of such fillers and/or hardeners include limestone, cellulose, sodium carbonate, and starch.

Preferably, the bonding resin according to the present invention does not contain formaldehyde. Preferably, the bonding resin does not contain phenol.

Preferably, the bonding resin according to the present invention does not contain basic catalyst.

Preferably, the bonding resin according to the present invention contains less than 1 wt-% lignin. More preferably the bonding resin according to the present invention does not contain lignin.

The aqueous solution of hydroxymethylfurfural (HMF), furfural (FU), furfuryl alcohol (FA), acetoxymethyl furfural or oligomer of HMF is preferably mixed with the crosslinker at room temperature, such as at a temperature of from 15° C. to 30° C. The mixing is preferably carried out for about 5 seconds to 2 hours. Preferably, the viscosity of the mixture is monitored during mixing, either continuously or by taking samples and determining the viscosity thereof.

In the production of mineral wool insulation, curing of the bonding resin to form an adhesive takes place when the components used for the preparation of the mineral wool insulation are exposed to heating. Curing may also take place without heating, such as at room temperature.

### EXAMPLES

### Example 1

Hydroxymethyl furfural (HMF) solution was prepared first by adding 105 g of Hydroxymethyl furfural and 243 g of water to a 1 L glass reactor at ambient temperature and stirred until the HMF was fully dissolved. Then, 52 g of 28-30% ammonia solution was added to the HMF solution. The composition was stirred for 60 minutes.

### Example 2

3-Aminopropyl trimethoxysilane was diluted to 1% solution in water. Binder composition was prepared by weighing 60 g of HMF-ammonia solution from the example 1, 6 g of Sorbitol polyglycidyl ether and 9 g of 1% of 3-aminopropyl trimethoxysilane into a 250 ml plastic container and was stirred with a wooden stick for 2 minutes. Then, 450 g silica sand was weighed into a bowl and the lignin mixture were poured on top of the sand and mixed for 2 minutes. Then, the sand bars were prepared by putting the sand-binder mixture into a silicon mould for baking in an oven at 200° C. for 1 hours. All sand bars were hard and stable after curing in the oven. The size of the bar for each test is height×thickness×length: 26 mm×18 mm×103 mm. Sand bars were post-cured for 24 hours and soaked in a water bath at 80° C. for 2 hours.

The sand bars were evaluated with 3-point bending test. The flexural strength before and after water soaking is given in the Table 1.

TABLE 1
Flexural Strength of the sand bars with and without conditioning

| | Flexural Strength | Flexural Strength after |
|---|---|---|
| | without conditioning | conditioning |
| | [MPa] | [MPa] |
| Sand bars | 9.7 | 5.6 |

In view of the above detailed description of the present invention, other modifications and variations will become apparent to those skilled in the art. However, it should be apparent that such other modifications and variations may be effected without departing from the spirit and scope of the invention.

1. A method for preparing a bonding resin, the method comprising:
mixing an aqueous solution comprising hydroxymethylfurfural, furfural, furfuryl alcohol, acetoxymethyl furfural, an oligomer of hydroxymethylfurfural, or a combination thereof with one or more crosslinkers selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, a diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate; and optionally with one or more additives.

2. The method according to claim 1, wherein the one or more crosslinkers are selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, and a diglycidyl ether of terminal diol having a linear carbon chain of 3-6 carbon atoms.

3. The method according to claim 2, wherein the one or more crosslinkers are selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, and a propylene glycol diglycidyl ether having 1-5 propylene glycol units.

4. The method according to claim 3, wherein the one or more crosslinkers comprises polyglycerol polyglycidyl ether.

5. The method according to claim 1, wherein the aqueous solution comprises at least 5% by weight of hydroxymethylfurfural, furfural, furfuryl alcohol, acetoxymethyl furfural or oligomer of hydroxymethylfurfural.

6. The method according to claim 1, wherein the one ore more additives are mixed with the aqueous solution, and wherein the one or more additives are selected from a group consisting of tannin, surfactant, dispersing agent, plasticizer, coupling agent, and/or a filler.

7. The method according to claim 1, wherein the aqueous solution has a pH in a range of from 6 to 14.

8. The method according claim 7, wherein the pH is in a range of from 10 to 14.

9. The method according to claim 1, wherein the aqueous solution further comprises ammonia, an organic base, or both.

10. A bonding resin obtained by the method according to claim 1.

11. A bonding resin comprising:
an aqueous solution comprising
hydroxymethylfurfural, furfural, furfuryl alcohol, acetoxymethyl furfural, an oligomer of hydroxymethylfurfural, or a combination thereof; and
one or more crosslinkers selected from a group consisting of: glycerol diglycidyl ether, polyglycerol diglycidyl ether, polyglycerol polyglycidyl ether, glycerol triglycidyl ether, sorbitol polyglycidyl ether, alkoxylated glycerol polyglycidyl ether, trimethylolpropane triglycidyl ether, trimethylolpropane diglycidyl ether, polyoxypropylene glycol diglycidylether, polyoxypropylene glycol triglycidyl ether, diglycidylether of cyclohexane dimethanol, resorcinol diglycidyl ether, isosorbide diglycidyl ether, pentaerythritol tetraglycidyl ether, ethylene glycol diglycidyl ether, polyethylene glycol diglycidyl ether having 2-9 ethylene glycol units, propylene glycol diglycidyl ether having 1-5 propylene glycol units, diglycidyl-, triglycidyl- or polyglycidyl-ether of a carbohydrate, diglycidyl-, triglycidyl- or polyglycidyl-ester of a carbohydrate, diglycidyl-ether or diglycidyl ester of salicylic acid, vanillic acid, or 4-hydroxybenzoic acid, an epoxidized or glycidyl substituted plant-based phenolic compound or epoxidized plant-based oil, tris(4-hydroxyphenyl) methane triglycidyl ether, N,N-bis(2,3-epoxypropyl)aniline, p-(2,3-epoxypropoxy-N,N-bis(2,3-epoxypropyl)aniline, diglycidyl ether of bis-hydroxymethylfuran, a diglycidyl ether of terminal diol having a linear carbon chain of 3-6 carbon atoms, and a crosslinker having functional groups selected from glycidyl amine, diglycidyl amine, triglycidyl amine, polyglycidyl amine, glycidyl amide, diglycidyl amide, triglycidyl amide, polyglycidyl amide, glycidyl ester, diglycidyl ester, triglycidyl ester, polyglycidyl ester, glycidyl azide, diglycidyl azide, triglycidyl azide, polyglycidyl azide, glycidyl methacrylate, diglycidyl methacrylate, triglycidyl methacrylate, or polyglycidyl methacrylate; and
optionally one or more additives.

12. A method for manufacturing a laminate, mineral wool insulation, or wood product, the method comprising:
preparing a bonding resin according to the method of claim 1; and,
manufacturing the laminate, mineral wool insulation, or wood product with the bonding resin.

13. The method according to claim 12, wherein the bonding resin is provided to a surface, and wherein curing of the bonding resin to form an adhesive takes place when the surface is exposed to pressure and heating.

14. A laminate, mineral wool insulation, or wood product comprising:
the bonding resin according to claim 11.
